SEQUENCE LISTING (20 sequences)

SEQ ID NO: 1 - DNA, 11 bp, Artificial Sequence
  Description of Artificial Sequence Synthetic oligonucleotide
  modified_base (6): arabinoguanosine
ctgtcnttctc

SEQ ID NO: 2 - DNA, 10 bp, Artificial Sequence
  Description of Artificial Sequence Synthetic oligonucleotide
  modified_base (5): arabinoguanosine
tgtcnttctc

SEQ ID NO: 3 - DNA, 9 bp, Artificial Sequence
  Description of Artificial Sequence Synthetic oligonucleotide
  modified_base (4): arabinoguanosine
gtcnttctc

SEQ ID NO: 4 - DNA, 8 bp, Artificial Sequence
  Description of Artificial Sequence Synthetic oligonucleotide
  modified_base (3): arabinoguanosine
tcnttctc

SEQ ID NO: 5 - DNA, 9 bp, Artificial Sequence
  Description of Artificial Sequence Synthetic oligonucleotide
  modified_base (3): 2′-deoxy-7-deazaguanosine
  modified_base (6): 2′-deoxy-7-deazaguanosine
tcntcnttg

SEQ ID NO: 6 - DNA, 8 bp, Artificial Sequence
  Description of Artificial Sequence Synthetic oligonucleotide
  modified_base (2): 2′-deoxy-7-deazaguanosine
  modified_base (5): 2′-deoxy-7-deazaguanosine
cntcnttg

SEQ ID NO: 7 - DNA, 7 bp, Artificial Sequence
  Description of Artificial Sequence Synthetic oligonucleotide
  modified_base (1): 2′-deoxy-7-deazaguanosine
  modified_base (4): 2′-deoxy-7-deazaguanosine
ntcnttg

SEQ ID NO: 8 - DNA, 6 bp, Artificial Sequence
  Description of Artificial Sequence Synthetic oligonucleotide
  modified_base (3): 2′-deoxy-7-deazaguanosine
tcnttg

SEQ ID NO: 9 - DNA, 11 bp, Artificial Sequence
  Description of Artificial Sequence Synthetic oligonucleotide
  misc_feature (5)..(6): phosphodiester linkage between bases 5 and 6
  modified_base (6): arabinoguanosine
ctgtcnttctc

SEQ ID NO: 10 - DNA, 10 bp, Artificial Sequence
  Description of Artificial Sequence Synthetic oligonucleotide
  misc_feature (4)..(5): phosphodiester linkage between bases 4 and 5
  modified_base (5): arabinoguanosine
tgtcnttctc

SEQ ID NO: 11 - DNA, 9 bp, Artificial Sequence
  Description of Artificial Sequence Synthetic oligonucleotide
  misc_feature (3)..(4): phosphodiester linkage between bases 3 and 4
  modified_base (4): arabinoguanosine
gtcnttctc

SEQ ID NO: 12 - DNA, 8 bp, Artificial Sequence
  Description of Artificial Sequence Synthetic oligonucleotide
  misc_feature (2)..(3): phosphodiester linkage between bases 2 and 3
  modified_base (3): arabinoguanosine
tcnttctc

SEQ ID NO: 13 - DNA, 11 bp, Artificial Sequence
  Description of Artificial Sequence Synthetic oligonucleotide
  modified_base (3): 2′-deoxy-7-deazaguanosine
  modified_base (7): 2′-deoxy-7-deazaguanosine
  modified_base (11): 2′-deoxy-7-deazaguanosine
tcnaacnttcn

SEQ ID NO: 14 - DNA, 10 bp, Artificial Sequence
  Description of Artificial Sequence Synthetic oligonucleotide
  modified_base (2): 2′-deoxy-7-deazaguanosine
  modified_base (6): 2′-deoxy-7-deazaguanosine
  modified_base (10): 2′-deoxy-7-deazaguanosine
cnaacnttcn

SEQ ID NO: 15 - DNA, 9 bp, Artificial Sequence
  Description of Artificial Sequence Synthetic oligonucleotide
  modified_base (1): 2′-deoxy-7-deazaguanosine
  modified_base (5): 2′-deoxy-7-deazaguanosine
  modified_base (9): 2′-deoxy-7-deazaguanosine
naacnttcn

SEQ ID NO: 16 - DNA, 8 bp, Artificial Sequence
  Description of Artificial Sequence Synthetic oligonucleotide
  modified_base (4): 2′-deoxy-7-deazaguanosine
  modified_base (8): 2′-deoxy-7-deazaguanosine
aacnttcn

SEQ ID NO: 17 - DNA, 11 bp, Artificial Sequence
  Description of Artificial Sequence Synthetic oligonucleotide
  modified_base (7): arabinoguanosine
tctgtcnttct

SEQ ID NO: 18 - DNA, 10 bp, Artificial Sequence
  Description of Artificial Sequence Synthetic oligonucleotide
  modified_base (6): arabinoguanosine
ctgtcnttct

SEQ ID NO: 19 - DNA, 9 bp, Artificial Sequence
  Description of Artificial Sequence Synthetic oligonucleotide
  modified_base (5): arabinoguanosine
tgtcnttct

SEQ ID NO: 20 - DNA, 8 bp, Artificial Sequence
  Description of Artificial Sequence Synthetic oligonucleotide
  modified_base (4): arabinoguanosine
gtcnttct

(19) United States
(12) Patent Application Publication
(10) Pub. No.: US 2007/0093439 A1
(43) Pub. Date: Apr. 26, 2007

(54) Short immunomodulatory oligonucleotides

(75) Inventors: Sudhir Agrawal, Shrewsbury, MA (US); Ekambar Kandimalla, Southboro, MA (US); Dong Yu, Westboro, MA (US); Lakshmi Bhagat, Framingham, MA (US)

Correspondence Address: Joseph C. Zucchero;Keown & Associates, Suite 1200, 500 West Cummings Park, Woburn, MA, 01801, US

(73) Assignee: Idera Pharmaceuticals, Inc.

(21) Appl. No.: 11/257,769

(22) Filed: Oct. 25, 2005

(51) Int. Cl. A61K 48/00; C07H 21/04

(52) U.S. Cl. 514/44; 536/23.1

(57) ABSTRACT

The invention relates to modulation of the immune system. More particularly, the invention relates to modulating the immune system through the use of oligonucleotide-derived compounds. The invention provides immunostimulatory agents that are less expensive to make than existing immunostimulatory oligonucleotides. The immunostimulatory agents according to the invention can, in preferred embodiments, cause immune stimulation across species lines.

### BACKGROUND OF THE INVENTION

1. Field of the Invention

The invention relates to modulation of the immune system. More particularly, the invention relates to modulating the immune system through the use of oligonucleotide-derived compounds.

2. Summar of the Related Art Tokunaga et al, J. Natl. Cancer Inst 72 :955-962 (1984); Messina et al., J. Immunol. 147: 1759-1764 (1991); Krieg et al., Nature 374: 546-549 (1995); Sato et al, Science 273 : 352-354 (1996), teach that the presence of CpG dinucleotides in certain sequence contexts in bacterial and synthetic oligodeoxyribonucleotides (CpG DNAs) are known to activate vertebrate innate immune reaction, T-cells and B cells.

Yamamoto et al., Jpn. J. Cancer Res. 79: 866-873 (1988); Halpern et al., Cell Immunol., 167: 72-78 (1996); Klinman et al., Proc. Natl. Acad. Sci. U.S.A. 93: 2879-2883 (1996); Zhao et al., Antisense Nucleic Acid Drug Dev. 7: 495-502 (1997) teach that the activation of immune cells by CpG DNA induces secretion of a number of cytokines, including IFN-γ, IL-12, TNF-α, and IL-6, and stimulates expression of costimulatory surface molecules.

Krieg et al., supra; Yamamoto et al, J. Immunol. 148; 4072-4076 (1992); Tokunaga et al., Microbiol. Immunol. 36: 55-66 (1992); Liang et al., J. Clin. Invest. 98: 1119-1129 (1996); Hartmann et al., J. Immunol. 164: 1617-1624 (2000), teach that the presence of a CpG dinucleotide and the sequences flanking the dinucleotide play a critical role in determining the immunostimulatory activity of DNA, that CpG dinucleotides in palindromic or non-palindromic hexameric sequences (P₁P₂CGP₃P₄) are required for immune stimulation, and further, that PuPuCGPyPy and PuTCG motifs optimally activate murine and human immune systems, respectively.

While these findings demonstrate that oligonucleotides are useful as immune stimulating agents, some problems with such use still exist. For example, long oligonucleotides are expensive to make and species specificity of flanking sequences limits the breadth of utility of any given oligonucleotide. There is, therefore, a need for less expensive immunostimulatory agents, and preferably immunostimulatory agents that have cross-species efficacy.

### BRIEF SUMMARY OF THE INVENTION

The invention provides immunostimulatory agents that are less expensive to make than existing immunostimulatory oligonucleotides. The immunostimulatory agents according to the invention can, in preferred embodiments, cause immune stimulation across species lines. Surprisingly, the present inventors have discovered that short oligonucleotide-based agents that are linked together with appropriate linkers can be made inexpensively and can be designed to cause immune stimulation in multiple species.

In a first aspect the invention provides an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

In a second aspect the invention provides pharmaceutical compositions. These compositions comprise any one of the compositions disclosed in the first aspect of the invention and a pharmaceutically acceptable carrier.

In a third aspect the invention provides a method for generating an immune response in a vertebrate, the method comprising administering to the vertebrate an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

In a fourth aspect the invention provides a method for therapeutically treating a vertebrate having cancer, an autoimmune disorder, airway inflammation, inflammatory disorders, skin disorders, allergy, asthma or a disease caused by a pathogen, such method comprising administering to the patient an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

In a fifth aspect the invention provides a method for preventing cancer, an autoimmune disorder, airway inflammation, inflammatory disorders, skin disorders, allergy, asthma or a disease caused by a pathogen in a vertebrate, such method comprising administering to the vertebrate an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

### BRIEF DESCRIPTION OF THE DRAWINGS

FIG. 1 shows cytokine secretion profiles of IMO-1 and its N-1, N-2, and N-3 analogs in mouse spleen cell cultures.

FIG. 2 shows cytokine secretion profiles of IMO-5 and its N-1, N-2, and N-3 analogs in mouse spleen cell cultures.

FIG. 3 shows cytokine secretion profiles of IMO-9 and its N-1, N-2, and N-3 analogs in mouse spleen cell cultures.

FIG. 4 shows cytokine secretion profiles of IMO- 13 and its N-1, N-2, and N-3 analogs in mouse spleen cell cultures.

### DETAILED DESCRIPTION OF THE PREFERRED EMBODIMENTS

The invention relates to modulation of the immune system. More particularly, the invention relates to modulating the immune system through the use of oligonucleotide-derived compounds. The patents and publications cited herein reflect the knowledge of those skilled in the art and are hereby incorporated by reference in their entirety. Any conflict between the teachings of such references and the instant specification shall be resolved in favor of the latter.

The invention provides immunostimulatory agents that are less expensive to make than existing immunostimulatory oligonucleotides. The immunostimulatory agents according to the invention can, in preferred embodiments, cause immune stimulation across species lines. Surprisingly, the present inventors have discovered that short oligonucleotide-based agents that are linked together with appropriate linkers can be made inexpensively and can be designed to cause immune stimulation in multiple species.

In a first aspect, the invention provides an immunostimulatory agent comprising two or more oligonucleotide branches linked together and having the structure: 5′-NₙpYpRpNₙp3′-Lₘ-3′NₙpRpYpNₙ-5′; wherein each N is independently selected from a nucleoside, a nucleoside analog; an arabinonucleoside, or an abasic sugar; each p is independently a natural or modified internucleoside linkage; at least one Y is selected from the group consisting of cytosine, 5-hydroxycytosine, N4-alkyl-cytosine, 4-thiouracil or other non-natural pyrimidine nucleoside or 2-oxo-7-deaza-8-methyl-purine, wherein when the base is 2-oxo-7-deaza-8-methyl-purine, it is covalently bound to the 1′-position of a pentose via the 1 position of the base; at least one R is selected from the group consisting of guanine, 2-amino-6-oxo-7-deazapurine, 2-amino-6-thiopurine, 6-oxopurine, or other non-natural purine nucleoside; L is a non-nucleotidic linker; each n is independently a number from 0-4, provided that neither branch exceeds 6 nucleotides; m is a number from 0-10 and wherein each N may optionally and independently be covalently linked to a non-nucleotidic linker. Preferred internucleoside linkages include phosphodiester, phosphorothioate and methylphosphonate linkages. The sequences of specific short oligonucleotide-based agents within these general structures used in the present study include, but are not limited to, those shown in Table 1.

TABLE 1

| | |
|---|---|
| SEQ ID NO: | Sequence |

| | |
|---|---|
| 1 | 5′-CTGTCR′TTCTC-X-CTCTTR′CTGTC-5′ |
| 2 | 5′-TGTCR′TTCTC-X-CTCTTR′CTGT-5′ |
| 3 | 5′-GTCR′TTCTC-X-CTCTTR′CTG-5′ |
| 4 | 5′-TCR′TTCTC-X-CTCTTR′CT-5′ |
| 5 | 5′-TCRTCRTTG-X-GTTRCTRCT-5′ |
| 6 | 5′-CRTCRTTG-X-GTTRCTRC-5′ |
| 7 | 5′-RTCRTTG-X-GTTRCTR-5′ |
| 8 | 5′-TCRTTG-X-GTTRCT-5′ |
| 9 | 5′-CTGTCoR′TTCTC-X-CTCTTR′oCTGTC-5′ |
| 10 | 5′-TGTCoR′TTCTC-X-CTCTTR′oCTGT-5′ |
| 11 | 5′-GTCoR′TTCTC-X-CTCTTR′oCTG-5′ |
| 12 | 5′-TCoR′TTCTC-X-CTCTTR′oCT-5′ |
| 13 | 5′-TCRAACRTTCR-X-TCTTR′CTGTCT-5′ |
| 14 | 5′-CRAACRTTCR-X-TCTTR′CTGTC-5′ |
| 15 | 5′-RAACRTTCR-X-TCTTR′CTGT-5′ |
| 16 | 5′-AACRTTCR-X-TCTTR′CTG-5′ |

R = 2′-deoxy-7-deazaguanosine; R′ = arabinoguanosine; X = glycerol linker; o = phosphodiester linkage

For purposes of the invention, a “non-nucleotidic linker” includes, without limitation a linker selected from a linker having a length of from about 2 angstroms to about 200 angstroms, C2-C18 alkyl linker, ethylene glycol linker, poly(ethylene glycol) linker, 2-aminobutyl-1,3-propanediol linker, glyceryl linker and branched alkyl linkers, acyclic alkyl linker, cyclic alkyl linker, aryl or heteroaryl linker, heterocyclic linker, polyalcohol linker, peptide linker, lipid linker and carbohydrate linker, each of which may be substituted or non-substituted.

For purposes of the invention, the term “oligonucleotide” refers to a polynucleoside formed from a plurality of linked nucleoside units. Such oligonucleotides can be obtained from existing nucleic acid sources, including genomic or cDNA, but are preferably produced by synthetic methods. In preferred embodiments each nucleoside unit includes a heterocyclic base and a pentofuranosyl, 2′-deoxypentfuranosyl, trehalose, arabinose, 2′-deoxy-2′-substituted arabinose, 2′-O-substituted arabinose or hexose sugar group. The nucleoside residues can be coupled to each other by any of the numerous known intemucleoside linkages. Such intemucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone intemucleoside linkages. The term “oligonucleotide” also encompasses polynucleosides having one or more stereospecific intemucleoside linkage (e.g., (Rₚ)- or (Sₚ)-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides, and oligonucleotides having backbone sections with alkyl linkers or amino linkers.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside an arabinonucleoside or a 2′-deoxy-2′-substituted-arabinoside.

For purposes of the invention, the term “2′-substituted ribonucleoside” or “2′-substituted arabinoside” includes ribonucleosides or arabinonucleoside in which the hydroxyl group at the 2′position of the pentose moiety is substituted to produce a 2′-substituted or 2′-O-substituted ribonucleoside. Preferably, such substitution is with a lower alkyl group containing 1-6 saturated or unsaturated carbon atoms, or with an aryl group having 6-10 carbon atoms, wherein such alkyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Examples of 2′-O-substituted ribonucleosides or 2′-O-substituted-arabinosides include, without linitation 2′-O-methylribonucleosides or 2′-O-methylarabinosides and 2′-O-methoxyethylribonucleosides or 2′-O-methoxyethylarabinosides.

The term “2′-substituted ribonucleoside” or “2′-substituted arabinoside” also includes ribonucleosides or arabinonucleosides in which the 2′-hydroxyl group is replaced with a lower alkyl group containing 1-6 saturated or unsaturated carbon atoms, or with an amino or halo group. Examples of such 2′-substituted ribonucleosides or 2′-substituted arabinosides include, without limitation, 2′-amino, 2′-fluoro, 2′-allyl, and 2′-propargyl ribonucleosides or arabinosides.

The term “oligonucleotide” includes hybrid and chimeric oligonucleotides. A “chimeric oligonucleotide” is an oligonucleotide having more than one type of internucleoside linkage. One preferred example of such a chimeric oligonucleotide is a chimeric oligonucleotide comprising a phosphorothioate, phosphodiester or phosphorodithioate region and non-ionic linkages such as alkylphosphonate or alkylphosphonothioate linkages (see e.g., Pederson et aL U.S. Pat. Nos. 5,635,377 and 5,366,878).

A “hybrid oligonucleotide” is an oligonucleotide having more than one type of nucleoside. One preferred example of such a hybrid oligonucleotide comprises a ribonucleotide or 2′-substituted ribonucleotide region, and a deoxyribonucleotide region (see, e.g., Metelev and Agrawal, U.S. Pat. Nos. 5,652,355, 6,346,614 and 6,143,881).

In a second aspect the invention provides pharmaceutical compositions. These compositions comprise any one of the compositions disclosed in the first aspect of the invention and a pharmaceutically acceptable carrier.

As used herein, the term “physiologically acceptable” refers to a material that does not interfere with the effectiveness of the compositions of the first, third, fourth or fifth aspects of the invention and is compatible with a biological system such as a cell, cell culture, tissue, or organism. In certain embodiments, the biological system is a living organism, such as a vertebrate.

As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990, ISBN: 0-912734-04-3.

The pharmaceutical compositions of the invention may be administered by any suitable route, including, without limitation, parenteral, oral, sublingual, transdermal, topical, intranasal, aerosol, intraocular, intratracheal, intrarectal, vaginal, by gene gun, dermal patch or in eye drop or mouthwash form. Administration of the therapeutic compositions of immunostimulatory oligonucleotides can be carried out using known procedures at dosages and for periods of time effective to reduce symptoms or surrogate markers of the disease. When administered systemically, the therapeutic composition is preferably administered at a sufficient dosage to attain a blood level of immunostimulatory oligonucleotide from about 0.0001 micromolar to about 10 micromolar. For localized administration, much lower concentrations than this may be effective, and much higher concentrations may be tolerated. Preferably, a total dosage of immunostimulatory oligonucleotide ranges from about 0.0001 mg per patient per day to about 200 mg per kg body weight per day. It may be desirable to administer simultaneously, or sequentially a therapeutically effective amount of one or more of the therapeutic compositions of the invention to an individual as a single treatment episode.

In a third aspect, the invention provides a method for generating an immune response in a vertebrate. The method according to this aspect of the invention comprises administering to the vertebrate an immunostimulatory oligonucleotide according to the first aspect of the invention. For purposes of the invention, the term “vertebrate” includes, without limitation, a fish, bird, or mammal. As used herein, the term “mammal” includes, without limitation rats, mice, cats, dogs, horses, cattle, cows, pigs, rabbits, non-human primates, and humans. “Modulating an immune response” means causing an increase or decrease in, or activation of one or more of B-cell induction, T-cell induction, cytokine induction, natural killer cell induction, specific cell surface marker expression, chemokine induction and activation of antigen presenting cells, such as dendritic cells, monocytes and macrophages.

In the method according to this aspect of the invention, administration of the immunostimulatory oligonucleotide can be alone or in a pharmaceutical composition and can be by any suitable route as described previously.

In a fourth aspect, the invention provides a method for treating a vertebrate having a disease or disorder. The method according to this aspect of the invention comprises administering to the vertebrate an immunostimulatory oligonucleotide according to the first aspect of the invention. The term “vertebrate” is as described previously. In the method according to this aspect of the invention, administration of the immunostimulatory oligonucleotide can be alone or in a pharmaceutical composition and can be by any suitable route as described previously.

In various embodiments, the disease or disorder to be treated is cancer, an autoimmune disorder, airway inflammation, inflammatory disorders, skin disorders, allergy, asthma or a disease caused by a pathogen. Pathogens include bacteria, parasites, fungi, viruses, viroids and prions.

In a fifth aspect, the invention provides a method for preventing cancer, an autoimmune disorder, airway inflammation, inflammatory disorders, skin disorders, allergy, asthma or a disease caused by a pathogen in a vertebrate. The method according to this aspect of the invention comprises administering to the vertebrate an immunostimulatory oligonucleotide according to the first aspect of the invention. The term “vertebrate” is as described previously. In the method according to this aspect of the invention, administration of the immunostimulatory oligonucleotide can be alone or in a pharmaceutical composition and can be by any suitable route as described previously.

In any of the methods according to the invention, the immunostimulatory oligonucleotide can be administered in combination with any other agent useful for treating the disease or condition that does not diminish the immunostimulatory effect of the oligonucleotide. For purposes of this aspect of the invention, the term “in combination with” means in the course of treating the same disease in the same patient, and includes administering the oligonucleotide and an agent in any order, including simultaneous administration, as well as any temporally spaced order, for example, from sequentially with one immediately following the other to up to several days apart. Such combination treatment may also include more than a single administration of the oligonucleotide, and independently the agent. The administration of the oligonucleotide and agent may be by the same or different routes.

In any of the methods according to the invention, the agent useful for treating the disease or condition includes, but is not limited to, vaccines, antigens, antibodies, cytotoxic agents, allergens, antibiotics, antisense oligonucleotides, chemotherapeutic agents, peptides, proteins, gene therapy vectors, DNA vaccines and/or adjuvants to enhance the specificity or magnitude of the immune response, or co-stimulatory molecules such as cytokines, chemokines, protein ligands, trans-activating factors, peptides and peptides comprising modified amino acids. Additionally, the agent can include DNA vectors encoding for antigen or allergen. In these embodiments, the oligonucleotides of the invention can variously act as adjuvants and/or produce direct immunostimulatory effects.

The following examples are provided to further illustrate certain particularly preferred embodiments of the invention and are not intended to limit the scope of the invention.

### EXAMPLE 1

### Synthesis and Purification of Oligomers

Immunostimulatory oligonucleotides were synthesized on a 1 to 2 lmole scale using β-cyanoethylphosphoramidites on a PerSeptive Biosystem's 8990 Expedite DNA synthesizer according to the manufacturer's directions. The phosphoramidites of dA, dG, dC, and T were obtained from PE Biosystems (Foster City, Calif.). C3-linker phosphoramidite was obtained from Glen Research Corporation (Sterling, Va.). Immunostimulatory oligonucleotides were synthesized on solid supports carrying DiDMT protected glyceryl linker obtained from ChemGenes (Wilmington, Mass.) using a parallel synthesis. Beaucage reagent (R.I. Chemicals, Orange, Calif.) was used as an oxidant to obtain the phosphorothioate backbone modification. Immunostimulatory oligonucleotides were deprotected using standard protocols, purified by HPLC, and dialyzed against USP quality sterile water for irrigation (Braun, Irving, Calif.). The immunostimulatory oligonucleotides were lyophilized and dissolved again in distilled water and the concentrations were determined from UV absorbance at 260 nm. All immunostimulatory oligonucleotides were characterized by CGE and MALDI-TOF mass spectrometry (Applied Biosystem's Voyager-DE™ STR Biospectrometry™ Workstation, Foster City, Calif.) for purity and molecular mass, respectively. The purity of full-length immunostimulatory oligonucleotides ranged from 89-95% with the rest being shorter by one or two nucleotides (n−1 and n−2) as determined by CGE and/or denaturing PAGE. All immunostimulatory oligonucleotides contained less than 0.075 EU/mL of endotoxin as determined by the Limulus assay (Bio-Whittaker, Walkersville, Md.).

### EXAMPLE 2

### Activity of Short-immunostimulatory Oligonucleotides in Murine Spleen Cell Cultures

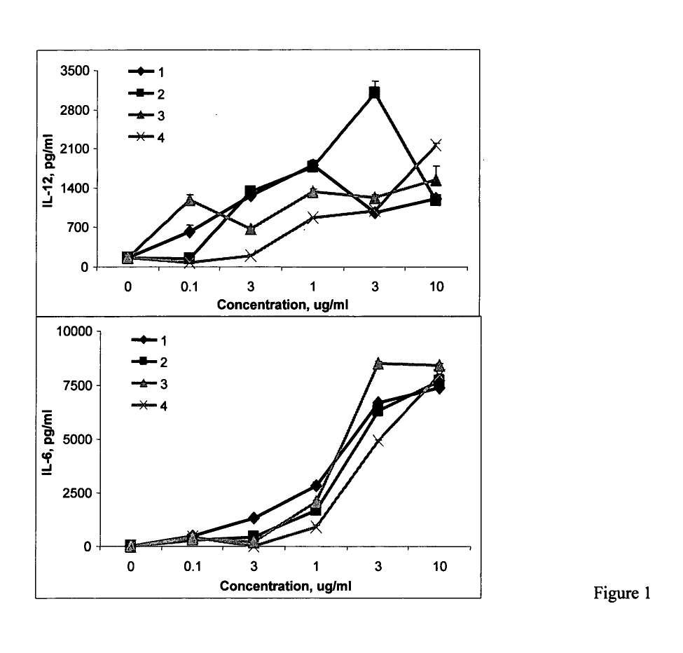

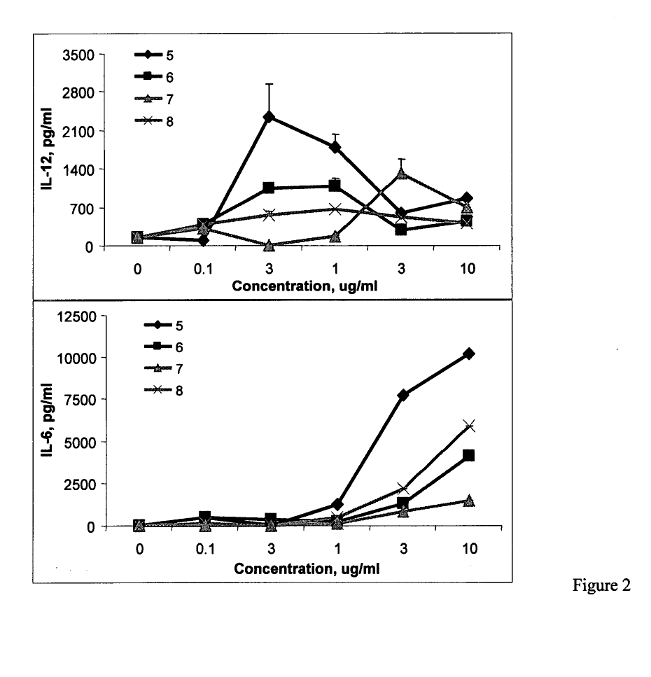

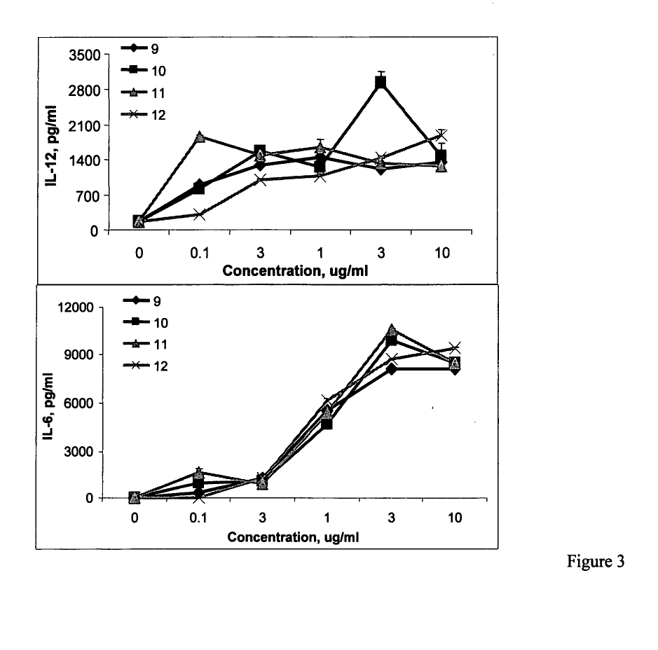

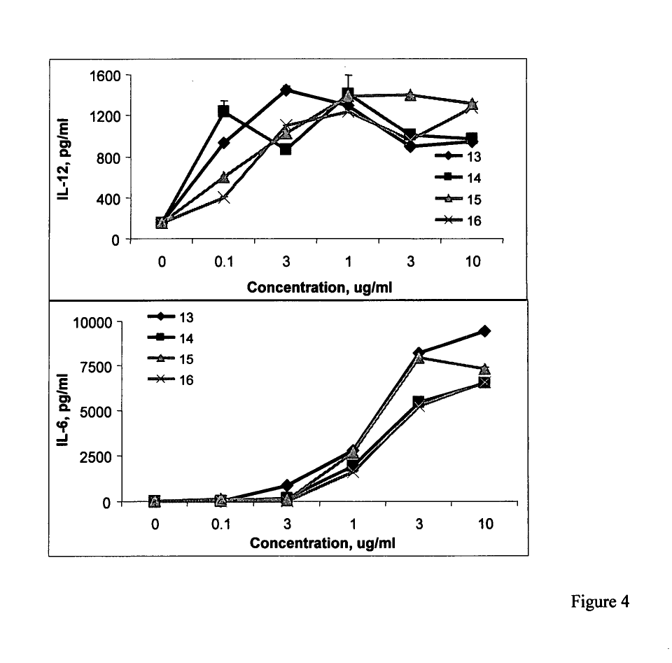

C57/ BL6 spleen cells were cultured with indicated concentrations of compounds. After 24 hours the supernatants were collected and the levels of IL-12 and IL-6 were determined by ELISA. All immunostimulatory oligonucleotides showed a concentration-dependent induction of two typical cytokines, IL-12 and IL-6 (FIGS. 1-4).

1. An immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

2. An immunostimulatory oligonucleotide according to claim 1 having the structure 5′-TGTCR′TTCTC-X-CTCTTR′CTGT-5′.

3. An immunostimulatory oligonucleotide according to claim 1 having the structure 5′-GTCR′TTCTC-X-CTCTTR′CTG-5′.

4. An immunostimulatory oligonucleotide according to claim 1 having the structure 5′-TCR′TTCTC-X-CTCTTR′CT-5′.

5. An immunostimulatory oligonucleotide according to claim 1 having the structure 5′-CRTCRTTG-X-GTTRCTRC-5′.

6. An immunostimulatory oligonucleotide according to claim 1 having the structure 5′-RTCRTTG-X-GTTRCTR-5′.

7. An immunostimulatory oligonucleotide according to claim 1 having the structure 5′-TCRTTG-X-GTTRCT-5′.

8. An immunostimulatory oligonucleotide according to claim 1 having the structure 5′-TGTCoR′TTCTC-X-CTCTTR′oCTGT-5′.

9. An immunostimulatory oligonucleotide according to claim 1 having the structure 5′-GTCoR′TTCTC-X-CTCTTR′oCTG-5′.

10. An immunostimulatory oligonucleotide according to claim 1 having the structure 5′-TCoR′TTCTC-X-CTCTTR′oCT-5′.

11. An immunostimulatory oligonucleotide according to claim 1 having the structure 5′-CRAACRTTCR-X-TCTTR′CTGTC-5′.

12. An immunostimulatory oligonucleotide according to claim 1 having the structure 5′-RAACRTTCR-X-TCTTR′CTGT-5′.

13. An immunostimulatory oligonucleotide according to claim 1 having the structure 5′-AACRTTCR-X-TCTTR′CTG-5′.

14. A pharmaceutical formulation comprising the oligonucleotide according to claim 1 and a physiologically acceptable carrier.

15. A method for generating an immune response in a vertebrate, the method comprising administering to the vertebrate an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

16. The method according to claim 15, wherein the route of administration is selected from parenteral, oral, sublingual, transdermal, topical, intranasal, aerosol, intraocular, intratracheal, intrarectal, vaginal, gene gun, dermal patch, eye drop and mouthwash.

17. The method according to claim 15 comprising administering an immunostimulatory oligonucleotide having the structure 5′-TGTCR′TTCTC-X-CTCTTR′CTGT-5′.

18. The method according to claim 15 comprising administering an immunostimulatory oligonucleotide having the structure 5′-GTCR′TTCTC-X-CTCTTR′CTG-5′.

19. The method according to claim 15 comprising administering an immunostimulatory oligonucleotide having the structure 5′-TCR′TTCTC-X-CTCTTR′CT-5′.

20. The method according to claim 15 comprising administering an immunostimulatory oligonucleotide having the structure 5′-CRTCRTTG-X-GTTRCTRC-5′.

21. The method according to claim 15 comprising administering an immunostimulatory oligonucleotide having the structure 5′-RTCRTTG-X-GTTRCTR-5′.

22. The method according to claim 15 comprising administering an immunostimulatory oligonucleotide having the structure 5′-TCRTTG-X-GTTRCT-5′.

23. The method according to claim 15 comprising administering an immunostimulatory oligonucleotide having the structure 5′-TGTCoR′TTCTC-X-CTCTTR′oCTGT-5′.

24. The method according to claim 15 comprising administering an immunostimulatory oligonucleotide having the structure 5′-GTCoR′TTCTC-X-CTCTTR′oCTG-5′.

25. The method according to claim 15 comprising administering an immunostimulatory oligonucleotide having the structure 5′-TCoR′TTCTC-X-CTCTTR′oCT-5′.

26. The method according to claim 15 comprising administering an immunostimulatory oligonucleotide having the structure 5′-CRAACRTTCR-X-TCTTR′CTGTC-5′.

27. The method according to claim 15 comprising administering an immunostimulatory oligonucleotide having the structure 5′-RAACRTTCR-X-TCTTR′CTGT-5′.

28. The method according to claim 15 comprising administering an immunostimulatory oligonucleotide having the structure 5′-AACRTTCR-X-TCTTR′CTG-5′.

29. A method for therapeutically treating a vertebrate having cancer, an autoimmune disorder, airway inflammation, inflammatory disorders, skin disorders, allergy, asthma or a disease caused by a pathogen, such method comprising administering to the patient an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

30. The method according to claim 29, wherein the route of administration is selected from parenteral, oral, sublingual, transdermal, topical, intranasal, aerosol, intraocular, intratracheal, intrarectal, vaginal, gene gun, dermal patch, eye drop and mouthwash.

31. The method according to claim 29 comprising administering an immunostimulatory oligonucleotide having the structure 5′-TGTCR′TTCTC-X-CTCTTR′CTGT-5′.

32. The method according to claim 29 comprising administering an immunostimulatory oligonucleotide having the structure 5′-GTCR′TTCTC-X-CTCTTR′CTG-5′.

33. The method according to claim 29 comprising administering an immunostimulatory oligonucleotide having the structure 5′-TCR′TTCTC-X-CTCTTR′CT-5′.

34. The method according to claim 29 comprising administering an immunostimulatory oligonucleotide having the structure 5′-CRTCRTTG-X-GTTRCTRC-5′.

35. The method according to claim 29 comprising administering an immunostimulatory oligonucleotide having the structure 5′-RTCRTTG-X-GTTRCTR-5′.

36. The method according to claim 29 comprising administering an immunostimulatory oligonucleotide having the structure 5′-TCRTTG-X-GTTRCT-5′.

37. The method according to claim 29 comprising administering an immunostimulatory oligonucleotide having the structure 5′-TGTCoR′TTCTC-X-CTCTTR′oCTGT-5′.

38. The method according to claim 29 comprising administering an immunostimulatory oligonucleotide having the structure 5′-GTCoR′TTCTC-X-CTCTTR′oCTG-5′.

39. The method according to claim 29 comprising administering an immunostimulatory oligonucleotide having the structure 5′-TCoR′TTCTC-X-CTCTTR′oCT-5′.

40. The method according to claim 29 comprising administering an immunostimulatory oligonucleotide having the structure 5′-CRAACRTTCR-X-TCTTR′CTGTC-5′.

41. The method according to claim 29 comprising administering an immunostimulatory oligonucleotide having the structure 5′-RAACRTTCR-X-TCTTR′CTGT-5′.

42. The method according to claim 29 comprising administering an immunostimulatory oligonucleotide having the structure 5′-AACRTTCR-X-TCTTR′CTG-5′.

43. A method for preventing cancer, an autoimmune disorder, airway inflammation, inflammatory disorders, skin disorders, allergy, asthma or a disease caused by a pathogen in a vertebrate, such method comprising administering to the vertebrate an immunostimulatory oligonucleotide having a structure from the group of 5′-TGTCR′TTCTC-X-CTCTTR′CTGT-5′, 5′-GTCR′TTCTC-X-CTCTTR′CTG-5′, 5′-TCR′TTCTC-X-CTCTTR′CT-5′, 5′-CRTCRTTG-X-GTTRCTRC-5′, 5′-RTCRTTG-X-GTTRCTR-5′, 5′-TCRTTG-X-GTTRCT-5′, 5′-TGTCoR′TTCTC-X-CTCTTR′oCTGT-5′, 5′-GTCoR′TTCTC-X-CTCTTR′oCTG-5′, 5′-TCoR′TTCTC-X-CTCTTR′oCT-5′, 5′-CRAACRTTCR-X-TCTTR′CTGTC-5′, 5′-RAACRTTCR-X-TCTTR′CTGT-5′, and 5′-AACRTTCR-X-TCTTR′CTG-5′; wherein wherein R=2′-deoxy-7-deazaguanosine; R′=arabinoguanosine; X=glycerol linker; and o=phosphodiester linkage.

44. The method according to claim 43, wherein the route of administration is selected from parenteral, oral, sublingual, transdermal, topical, intranasal, aerosol, intraocular, intratracheal, intrarectal, vaginal, gene gun, dermal patch, eye drop and mouthwash.

45. The method according to claim 43 comprising administering an immunostimulatory oligonucleotide having the structure 5′-TGTCR′TTCTC-X-CTCTTR′CTGT-5′.

46. The method according to claim 43 comprising administering an immunostimulatory oligonucleotide having the structure 5′-GTCR′TTCTC-X-CTCTTR′CTG-5′.

47. The method according to claim 43 comprising administering an immunostimulatory oligonucleotide having the structure 5′-TCR′TTCTC-X-CTCTTR′CT-5′.

48. The method according to claim 43 comprising administering an immunostimulatory oligonucleotide having the structure 5′-CRTCRTTG-X-GTTRCTRC-5′.

49. The method according to claim 43 comprising administering an immunostimulatory oligonucleotide having the structure 5′-RTCRTTG-X-GTTRCTR-5′.

50. The method according to claim 43 comprising administering an immunostimulatory oligonucleotide having the structure 5′-TCRTTG-X-GTTRCT-5′.

51. The method according to claim 43 comprising administering an immunostimulatory oligonucleotide having the structure 5′-TGTCoR′TTCTC-X-CTCTTR′oCTGT-5′.

52. The method according to claim 43 comprising administering an immunostimulatory oligonucleotide having the structure 5′-GTCoR′TTCTC-X-CTCTTR′oCTG-5′.

53. The method according to claim 43 comprising administering an immunostimulatory oligonucleotide having the structure 5′-TCoR′TTCTC-X-CTCTTR′oCT-5′.

54. The method according to claim 43 comprising administering an immunostimulatory oligonucleotide having the structure 5′-CRAACRTTCR-X-TCTTR′CTGTC-5′.

55. The method according to claim 43 comprising administering an immunostimulatory oligonucleotide having the structure 5′-RAACRTTCR-X-TCTTR′CTGT-5′.

56. The method according to claim 43 comprising administering an immunostimulatory oligonucleotide having the structure 5′-AACRTTCR-X-TCTTR′CTG-5′.

57. (canceled)

58. The pharmaceutical composition according to claim 14, further comprising an antibody, antisense oligonucleotide, protein, antigen, allergen, chemotherapeutic agent or adjuvant.

59. The method according to claim 15, further comprising administering an antibody, antisense oligonucleotide, protein, antigen, allergen, chemotherapeutic agent or adjuvant.

60. The method according to claim 29, further comprising administering an antibody, antisense oligonucleotide, protein, antigen, allergen, chemotherapeutic agent or adjuvant.

61. The method according to claim 43, further comprising administering an antibody, antisense oligonucleotide, protein, antigen, allergen, chemotherapeutic agent or adjuvant.
